(19) United States
(12) Patent Application Publication
(10) Pub. No.: US 2022/0323315 A1
(43) Pub. Date: Oct. 13, 2022

(54) OIL-IN-WATER TYPE EMULSION LIP COSMETIC

(71) Applicant: Shiseido Company, Ltd., Tokyo (JP)

(72) Inventors: Misa KANAZAWA, Tokyo (JP); Yurie NISHIUMI, Tokyo (JP); Kiriko CHIBA, Tokyo (JP)

(73) Assignee: Shiseido Company, Ltd., Tokyo, (JP)

(21) Appl. No.: 17/615,967

(22) PCT Filed: May 28, 2020
(86) PCT No.: PCT/JP2020/021037

§ 371 Date: Dec. 2, 2021

(30) Foreign Application Priority Data
Jun. 3, 2019 (JP) 2019-103783

(51) Int. Cl. A61K 8/06; A61K 8/25; A61K 8/60; A61K 8/37; A61K 8/34; A61K 8/81; A61K 8/31; A61Q 1/04

(52) U.S. Cl. CPC A61K 8/062; A61K 8/25; A61K 8/60; A61K 8/37; A61K 8/342; A61K 8/8111; A61K 8/31; A61Q 1/04; A61K 2800/43; A61K 2800/596

(57) ABSTRACT

The present invention provides an oil-in-water emulsion lip cosmetic that has no whiteness, and is excellent in watery feel and high-temperature stability.

The oil-in-water emulsion lip cosmetic comprising the following (A) to (D), and having a viscosity of 7500 to 50000 mPa·s.
(A) An oil component comprising (a1) and (a2)
   (a1) 5 to 70% by mass of methylphenylsilicone
   (a2) 5 to 70% by mass of an oil component that separates when mixed with (a1) at 25° C.
(B) An aqueous component comprising (b1) and (b2)
   (b1) 10 to 80% by mass of an aqueous component
   (b2) 0.01 to 10% by mass of a colorant
(C) A surfactant comprising (c1) and (c2)
   (c1) 2.5% by mass or greater of a surfactant that dissolves 1% by mass or more relative to (a2) when mixed with (a2) at 25° C.
   (c2) 0 to 0.5% by mass of a sucrose fatty acid ester
(D) A higher alcohol comprising (d1)
   (d1) 0.5% by mass or greater of a higher alcohol having a branched chain and 6 or more carbon atoms in the total amount of the higher alcohol.

### RELATED APPLICATIONS

This application claims the priority of Japanese Patent Application No. 2019-103783 filed on Jun. 3, 2019, which is incorporated herein by reference.

### TECHNICAL FIELD

The present invention relates to an oil-in-water emulsion lip cosmetic, and particularly to an oil-in-water emulsion lip cosmetic that has no whiteness, and is excellent in watery feel and high-temperature stability.

### BACKGROUND ART

In conventional lip cosmetics, a large amount of oil components is blended to impart glossy appearance. In addition, colorants are often blended to the oil components to impart color. However, when colorants are blended to the oil components, it was disadvantageous in the point that transparency of the oil components deteriorates.

Therefore, in order to impart watery feel to lip cosmetics without deteriorating transparency of oil components, oil-in-water emulsion lip cosmetics that comprise colorants in an aqueous phase have been developed. For example, an oil-in-water-lip cosmetic that comprises an oil component such as a non-volatile hydrocarbon oil including a hydrogenated polyisobutene, and an aqueous phase that comprises a colorant is known (Patent Literature 1). However, this oil-in-water lip cosmetic may become whitish, or the viscosity may be relatively high; therefore, there was a room for improvement in watery feel.

### CITATION LIST

[Patent Literature 1] Japanese Unexamined Patent Publication No. 2018-2626 A

### SUMMARY OF INVENTION

### Technical Problem

The present invention was made in view of the above-described conventional art. An object of the invention is to provide an oil-in-water emulsion lip cosmetic that has no whiteness, and is excellent in watery feel and high-temperature stability.

### Solution to Problem

As a result of the present inventors' earnest study to solve the problems, it was found that an oil-in-water emulsion lip cosmetics that has no whiteness and is excellent in watery feel and high-temperature stability can be obtained by comprising, at specific amounts: (A) an oil component comprising methylphenylsilicone (a1), and an oil component (a2) that separates when mixed with (a1) at 25° C.; (B) an aqueous component comprising an aqueous component (b1) and a colorant (b2); (C) a surfactant (c1) that dissolves when mixed with (a2) 25° C.; and (D) a higher alcohol (d1) comprising an alcohol having a branched chain and 6 or more carbon atoms. Based on the findings, the present invention has been completed.

That is, the oil-in-water emulsion lip cosmetic according to the present invention comprises the following (A) and (B), and has a viscosity of 7500 to 50000 mPa·s.

(A) An oil component comprising (a1) and (a2)
(a1) 5 to 70% by mass of methylphenylsilicone
(a2) 5 to 70% by mass of an oil component that separates when mixed with (a1) at 25° C.
   (B) An aqueous component comprising (b1) and (b2)
(b1) 10 to 80% by mass of an aqueous component
(b2) 0.01 to 10% by mass of a colorant
   (C) A surfactant comprising (c1) and (c2)
(c1) 2.5% by mass or greater of a surfactant that dissolves 1% by mass or more relative to (a2) when mixed with (a2) at 25° C.
(c2) 0 to 0.5% by mass of a sucrose fatty acid ester
   (D) A higher alcohol comprising (d1)
(d1) 0.5% by mass or greater of a higher alcohol having a branched chain and 6 or more carbon atoms in the total amount of the higher alcohol.

In the cosmetic, the oil component (a2) that separates when mixed with (a1) at 25° C. is preferably selected from a group consisting of a hydrogenated polyisobutene, hydrogenated polydecene, vaseline, pentaerythritol ester, liquid paraffin, squalane, polyethylene wax, and microcrystalline wax.

In the cosmetic, the alcohol (d1) having a branched chain and 6 or more carbon atoms is preferably isostearyl alcohol.

In the cosmetic, the surfactant (c1) that dissolves when mixed with (a2) at 25° C. is preferably selected from a group consisting of sorbitan monostearate, polyoxyethylene sorbitan fatty acid ester, polyoxyethylene behenyl ether, PEG-40 stearate, PEG-55 stearate, glyceryl stearate, sorbitan sesquioleate, and PEG-5 glyceryl stearate.

### Effect of Invention

The oil-in-water emulsion lip cosmetic according to the present invention comprises, at specific amounts: (A) an oil component comprising methylphenylsilicone (a1), and an oil component (a2) that separates when mixed with (a1) at 25° C.; (B) an aqueous component comprising an aqueous component (b1) and a colorant (b2); (C) a surfactant (c1) that dissolves when mixed with (a2) at 25° C.; and (D) a higher alcohol (d1) comprising an alcohol having a branched chain and 6 or more carbon atoms. The oil-in-water emulsion lip cosmetic that has no whiteness, and is excellent in watery feel and high-temperature stability can be provided.

### DESCRIPTION OF EMBODIMENTS

((A) Oil Phase)

The oil phase (A) needs to comprise, at specific amounts, methylphenylsilicone (a1) and an oil component (a2) that separates when mixed with (a1) at 25° C.

Examples of methylphenylsilicone (a1) include diphenyl dimethicone, trimethyl pentaphenyl trisiloxane, diphenylsiloxy phenyl trimethicone and phenyl trimethicone.
Among them, it is preferred to comprise dimethyl dimethicone.

The blending amount of methylphenylsilicone (a1) needs to be 5 to 70% by mass relative to the total amount of the cosmetic. It is preferably 10% by mass or greater, and more preferably 15% by mass or greater. When the blending amount of the component (a1) is too small, separation upon application may become difficult, and an effect of maintaining glossy appearance may not be exhibited. In addition, it is preferably 65% by mass or less. When the blending amount is too large, the blending amount of the component (a2) becomes too small; therefore, adhesion to lips may deteriorate, and thus the effect of maintaining glossy appearance may not be exhibited.

The oil component (a2) that separates when mixed with (a1) at 25° C. is an oil component that adheres to lips and imparts glossy appearance after the aqueous component (b1) is evaporated after application of the lip cosmetic.

“Separation” was measured with the following condition.

### (Measurement Condition)

(a1) and (a2) were used at (a1):(a2)=1:1 (mass ratio), heated to 90° C., mixed with stirring, and left to stand still. When the mixture became 25° C., one that had a uniform boundary and was separated into two phases was determined as “separated”, and one that was in a semi-transparent state or a transparent compatible state without a boundary was determined as “not separated”.

Examples of the component (a2) include a hydrogenated polyisobutene, hydrogenated polydecene, Vaseline, pentaerythritol ester, liquid paraffin, squalane, polyethylene wax, and microcrystalline wax.

Examples of pentaerythritol ester include pentaerythrityl tetrabenzoate/2-ethylhexanoate, pentaerythrityl tetrabehenate/benzoate/2-ethylhexanoate, pentaerythrityl tetra-2-ethylhexanoate, and dipentaerythrityl hexa(12-hydroxystearate).

These oil components need to be blended at a ratio that satisfies the above separation condition as the entire component (a2).

The blending amount of the oil component (a2) that separates when mixed with (a1) at 25° C. needs to be 5 to 70% by mass relative to the total amount of the cosmetic. It is preferably 10% by mass or greater, and more preferably 15% by mass or greater. When the blending amount of the component (a2) is too small, phase separation of the components (a1) and (a2) may become difficult. In addition, it is preferably 65% by mass or less. When the blending amount is too large, the blending amount of the component (a1) may be too small, and thus glossy appearance may be poor.

Even when oil components other than the components (a1) and (a2) are mixed, the state of phase separation of the component (a1) and the component (a2) can be maintained as long as they are within a specific range. Accordingly, other oil components can be blended within a range that can maintain the phase-separated state, and a range of not deteriorating glossy appearance.

Examples of such oil components include behenyl/phytosteryl dimer dilinoleate, glyceryl diisostearate, trimethylolpropane tri-2-ethylisostearate, isopropyl myristate, cetyl-2-ethylhexanoate, glyceryl triisostearate, 2-heptylundecyl palmitate, methyl polysiloxane, glycerin triisostearate, diisostearyl malate, phytosteryl/octyldodecyl lauroyl glutamate, cyclopentasiloxane, diisostearyl malate, isododecane, triethylhexanoin, glyceryl diisostearate, trimethylolpropane triethylhexanoate, and triisostearin.

((B) Aqueous Phase)

The aqueous phase (B) needs to comprise the aqueous component (b1) and the colorant (b2) at specific amounts.

The aqueous component (b1) can be blended with components that are commonly blended to cosmetics.

Examples of the aqueous component (b1) include water, water-swellable thickeners, moisturizers, preservatives, dispersants, pH adjusters, and antifoaming agents.

The blending amount of the aqueous component (b1) needs to be 10 to 80% by mass relative to the total amount of the cosmetic. It is preferably 15% by mass or greater. When the blending amount of the component (b1) is less than 10% by mass, feeling in use such as watery feel may be poor, or it may become difficult to blend the colorant (b2). It is preferably 60% by mass or less. When it exceeds 80% by mass, the amount of the oil phase (A) may be too small, and thus glossy appearance may be poor.

Since transparency of the oil phase is deteriorated when the colorant (b2) is blended to the oil phase, the colorant (b2) needs to be blended to the aqueous phase. When it is blended to the oil phase, transparency of the oil phase deteriorates.

Examples of the colorant (b2) include water-soluble dyes, inorganic pigments and organic pigments.

Examples of water-soluble dyes include Red No. 227, Yellow No. 4, Yellow No. 5, and Red No. 230(1).

Examples of inorganic pigments include: inorganic powders such as talc, kaolin, mica, sericite, muscovite, biotite, phlogopite, synthetic mica, silica, zeolite, barium sulphate, calcined calcium sulphate, calcined gypsum, calcium phosphate, fluorine apatite, hydroxyapatite, and ceramic powder; inorganic white pigments such as titanium dioxide and zinc oxide; inorganic red pigments such as iron oxide (Bengala) and iron titanate; inorganic brown pigments such as γ-iron oxide; inorganic yellow pigments such as yellow iron oxide and ocher; inorganic black pigments such as black iron oxide, carbon, and lower titanium oxide; inorganic purple pigments such as mango violet and cobalt violet; inorganic green pigments such as chromium oxide, chromium hydroxide, and cobalt titanate; inorganic blue pigments such as ultramarine and Prussian blue; and pearl pigments such as titanium oxide coated mica, titanium oxide coated bismuth oxychloride, titanium oxide coated talc, colored titanium oxide coated mica, bismuth oxychloride, and fish scale flakes.

Examples of organic pigments include zirconium, barium and aluminum lakes such as Red No. 202, Red No. 205, Red No. 220, Red No. 228, Red No. 405, Orange No. 203, Orange No. 204, Yellow No. 205, Yellow No. 401, Blue No. 404, Red No. 3, red No. 104, Red No. 227, Red No. 401, Orange No. 205, Yellow No. 4, Yellow No. 202, Green No. 3, and Blue No. 1.

The blending amount of the colorant (b2) needs to be 0.01 to 10% by mass relative to the total amount of the cosmetic. It is preferably 0.1% by mass or greater. When the blending amount of the component (b2) is less than 0.01% by mass, lips may not be colored sufficiently. It is preferably 5% by mass or less. When it exceeds 10% by mass, watery feel may be poor.

((C) Surfactant Comprising (c1) and (c2))

The surfactant (C) comprising (c1) and (c2) needs to comprise a surfactant (c1), at a specific amount, that dissolves 1% by mass or greater relative to the component (a2) when mixed with the component (a2) at 25° C. When sucrose fatty acid ester (c2) is blended, it needs to be 0.3% by mass or less; however, it is preferred not to blend it.

The surfactant (c1) that dissolves when mixed with the component (a2) at 25° C. is a surfactant that dissolves when mixed with the component (a2) at a mass ratio of 1:1 at 25° C.

Examples thereof include: polyoxyethylene sorbitan fatty acid esters such as sorbitan monostearate, POE (20) sorbitan monostearate, POE (20) sorbitan monolaurate, and POE (20) sorbitan oleate; polyoxyethylene behenyl ethers such as POE (20) behenyl ether, POE (10) behenyl ether, and POE (30) behenyl ether; PEG-40 stearate; PEG-55 stearate; glyceryl stearate; sorbitan sesquiisostearate; and PEG-5 glyceryl stearate.

Among them, it is preferred to use sorbitan monostearate, POE (20) sorbitan monostearate, or POE (20) behenyl ether.

The blending amount of the surfactant (c1) that dissolves when mixed with the component (a2) at 25° C. needs to be 2.5 to 5.8% by mass relative to the total amount of the cosmetic. It is preferably 4% by mass or greater, and more preferably 4.5% by mass or greater. When the blending amount of the surfactant is too small, high-temperature stability may be poor. When the blending amount of the surfactant is too large, feel upon use may be poor due to an increase of viscosity.

The blending amount of sucrose fatty acid ester (c2) needs to be 0 to 0.5% by mass relative to the total amount of the cosmetic. It is preferably 0.1% by mass or less, and it is more preferred not to comprise it. By blending sucrose fatty acid ester, whiteness may occur to application color; therefore, transparency may be poor.

((D) Higher Alcohol Comprising (Dl))

The component (D) needs to comprise an alcohol having a branched chain and 6 or more carbon atoms at a specific amount.

Examples of the alcohol (d1) having a branched chain and 6 or more carbon atoms include isostearyl alcohol, octyldodecanol, hexyldecanol, and decyltetradecanol.

In oil-in-water cosmetics, the higher alcohol is usually added to the oil phase; however, in the oil-in-water emulsion lip cosmetic of the present invention, it is preferred to add it to the aqueous phase.

The blending amount of the alcohol (d1) having a branched chain and 6 or more carbon atoms is preferably 0.5% by mass or greater, and more preferably 1% by mass or greater. When the blending amount is too small, high-temperature stability may be poor.

The viscosity of the oil-in-water emulsion lip cosmetic according to the present invention needs to be within a range of 7500 to 50000 mPa·s. The viscosity is preferably 10000 mPa·s or greater, and more preferably 15000 mPa·s or greater. When the viscosity is too low, high-temperature stability may be poor. The viscosity is preferably 30000 mPa·s or less. When the viscosity is too high, watery feel may be poor.

The oil-in-water emulsion lip cosmetic of the present invention may be applied to liquid lip cosmetics such as a lip gloss, solid lip sticks, lip serums, and lip concealers.

### Examples

The present invention will be further described in the following examples. However, the present invention is not limited by these examples. In the following examples, the blending amount of each component will be expressed with “% by mass” unless otherwise specified.

First, the evaluation methods used in the present invention will be described.

### Evaluation (1): Viscosity

The viscosity was measured with a B-type viscometer at ambient temperature

### Evaluation (2): Whiteness

One expert panel applied the sample to one's lip, and evaluated whiteness of the lip immediately after application.

### (Evaluation Criteria)

S: There was no whiteness.
A: There was slight whiteness.
B: There was outstanding whiteness.
C: There was significantly outstanding whiteness.

### Evaluation (3): Watery Feel

One expert panel applied the sample to one's lip, and evaluated watery feel immediately after application.

### (Evaluation Criteria)

S: There was significant watery feel.
A: There was watery feel.
B: There was no watery feel, and was sticky.

### Evaluation (4): High-Temperature Stability

A: Coalescence of emulsion particles was not observed at all at high temperature.
B: Coalescence of emulsion particles was slightly observed at high temperature.
C: Coalescence of emulsion particles was remarkably observed at high temperature.

As for high-temperature stability, evaluation of B or greater was regarded as a suitable range.

The inventors prepared the oil-in-water emulsion lip cosmetics as shown in the following Table 1 with a common method, and evaluated the cosmetics based on the above evaluation criteria (1) to (4). The results are shown in Table 1.

TABLE 1

| Test example | | | 1-1 | 1-2 | 1-3 | 1-4 | 1-5 | 1-6 | 1-7 | 1-8 | 1-9 |
|---|---|---|---|---|---|---|---|---|---|---|---|
| (A) | (a1) | Diphenyl dimethicone | 18 | 18 | 18 | 18 | 18 | 18 | 18 | 18 | 18 |
| | (a2) | Hydrogenated polyisobutene | 27.54 | 27.54 | 27.54 | 27.54 | 27.54 | 27.54 | 27.54 | — | 27.54 |
| | | Hydrogenated polydecene | — | — | — | — | — | — | — | 27.54 | — |
| | | Vaseline | 10 | 10 | 10 | 10 | 10 | 10 | 10 | 10 | 10 |
| | | Phytosteryl/behenyl dimer | 10 | 10 | 10 | 10 | 10 | 10 | 10 | 10 | 10 |
| | | dilinoleate | | | | | | | | | |
| (B) | (b1) | Ion-exchanged water | Balance | Balance | Balance | Balance | Balance | Balance | Balance | Balance | Balance |
| | | Dipropylene glycol | 5 | 5 | 5 | 5 | 5 | 5 | 5 | 5 | 5 |
| | | Ethanol | 1.5 | 1.5 | 1.5 | 1.5 | 1.5 | 1.5 | 1.5 | 1.5 | 1.5 |
| | | Phenoxy ethanol | 0.25 | 0.25 | 0.25 | 0.25 | 0.25 | 0.25 | 0.25 | 0.25 | 0.25 |
| | | Sodium metaphosphate | 0.05 | 0.05 | 0.05 | 0.05 | 0.05 | 0.05 | 0.05 | 0.05 | 0.05 |
| | | Polyvinyl alcohol | 0.25 | 0.25 | 0.25 | 0.25 | 0.25 | 0.25 | 0.25 | 0.25 | 0.25 |
| | | Bentonite | 0.3 | 0.3 | 0.3 | 0.3 | 0.3 | 0.3 | 0.3 | 0.3 | 0.3 |
| | (b2) | Red No. 202 | 1 | 1 | 1 | 1 | 1 | 1 | 1 | 1 | 1 |
| | | Titanium oxide | 1 | 1 | 1 | 1 | 1 | 1 | 1 | 1 | 1 |
| (C) | (c1) | Sorbitan monostearate | 0.86 | 0.86 | 0.86 | 0.86 | 0.86 | 0.86 | 0.86 | 0.86 | 0.86 |
| | | Polysorbate-60 | 1.3 | 1.3 | 2 | 3 | 3 | 3 | 3 | 3 | 4.7 |
| | | Beheneth-20 | — | — | — | — | 1.7 | 1.7 | 1.7 | 1.7 | — |
| | (c2) | Sucrose fatty acid ester | 2.2 | 0.5 | — | — | — | — | — | — | — |
| (D) | (d1) | Isostearyl alcohol | — | — | — | — | — | — | 1 | 1 | — |
| | | Behenyl alcohol | 1 | 1 | 1 | 1 | 1 | — | — | — | 1 |
| | | Cetanol | — | — | — | — | — | 1 | — | — | — |

| | | | | | | | | | |
|---|---|---|---|---|---|---|---|---|---|
| Viscosity (mPa · s) | 61300 | 18500 | 11600 | 17900 | 48000 | 42500 | 12200 | 26600 | 39000 |
| Whiteness | B | A | S | S | S | S | S | S | S |
| Watery feel | B | S | S | S | A | A | S | S | A |
| High-temperature stability | A | C | C | B | A | A | A | A | B |

(*1): Deodorizing polybutene P (manufactured by NIKKO RICA CORPORATION)
(*2): Olefin oligomer 30 (manufactured by Nikko Chemicals Co., Ltd.)
(*3): Plandool-PB (manufactured by NIPPON FINE CHEMICAL CO., LTD.)
(*4): NIKKOL SS-10V (manufactured by Nikko Chemicals Co., Ltd.)
(*5): NIKKOL TS-10V (manufactured by Nikko Chemicals Co., Ltd.)
(*6): NIKKOL BB-20 (manufactured by Nikko Chemicals Co., Ltd.)
(*7): DK ESTER S-160N (manufactured by DKS Co., Ltd.)

According to Test example 1-1, it was found that when the blending amount of sucrose fatty acid aster in the oil-in-water emulsion lip cosmetic is large, it was excellent in high-temperature stability, but poor in whiteness and watery feel.

By reducing the blending amount of sucrose fatty acid aster, the oil-in-water emulsion lip cosmetic having excellent whiteness and watery feel was obtained; however, it was poor in high-temperature stability (Test examples 1-2, 1-3).

By increasing the blending amount of the surfactant (c1) that dissolves when mixed with the component (a2) at 25° C., it was found that high-temperature stability could be improved; however, watery feel deteriorated (Test examples 1-5, 1-6).

By substituting the higher alcohol to be blended to an alcohol having a branched chain and 6 or more carbon atoms, it was found that watery feel could be improved without deteriorating high-temperature stability, and also the viscosity could be lowered (Test examples 1-7, 1-8).

In the present invention, by using at least two types or more, more preferably three types, of sorbitan fatty acid ester-based surfactants, fatty acid polyoxyethylene sorbitan-based surfactants (Polysorbate 60, or the like), and polyoxyethylene behenyl ether-based surfactants (Beheneth-20, or the like) as the surfactant (c1), particularly excellent watery feel and high-temperature stability can be achieved.

Accordingly, in the oil-in-water emulsion lip cosmetic according to the present invention, it is necessary to comprise specific amounts of: (A) the oil component comprising methylphenylsilicone (a1) and the oil component (a2) that separates when mixed with methylphenylsilicone (a1) at 25° C.; (B) the aqueous component comprising the aqueous component (b1) and the colorant (b2); (C) the surfactant that dissolves when mixed with the component (a2) at 25° C.; and (D) the higher alcohol comprising the alcohol (d1) having a branched chain and 6 or more carbon atoms.

The inventors prepared the oil-in-water emulsion lip cosmetics as shown in the following Table 2 with a common method, and evaluated the cosmetics based on the above evaluation criteria (1) to (4). The results are shown in Table 2.

TABLE 2

| Test example | | | 2-1 | 1-7 | 2-2 | 2-3 |
|---|---|---|---|---|---|---|
| (A) | (a1) | Diphenyl dimethicone | 18 | 18 | 18 | 18 |
| | (a2) | Hydrogenated polyisobutene | 27.54 | 27.54 | 27.54 | 27.54 |
| | | Vaseline | 10 | 10 | 10 | 10 |
| | | Phytosteryl/behenyl dimer dilinoleate | 10 | 10 | 10 | 10 |
| (B) | (b1) | Ion-exchanged water | Balance | Balance | Balance | Balance |
| | | Dipropylene glycol | 5 | 5 | 5 | 5 |
| | | Ethanol | 1.5 | 1.5 | 1.5 | 1.5 |
| | | Phenoxy ethanol | 0.25 | 0.25 | 0.25 | 0.25 |
| | | Sodium metaphosphate | 0.05 | 0.05 | 0.05 | 0.05 |
| | | Polyvinyl alcohol | 0.25 | 0.25 | 0.25 | 0.25 |
| | | Bentonite | 0.3 | 0.3 | 0.3 | 0.3 |
| | (b2) | Red No. 202 | 1 | 1 | 1 | 1 |
| | | Titanium oxide | 1 | 1 | 1 | 1 |
| (C) | (c1) | Sorbitan monostearate | 0.43 | 0.86 | 0.92 | 0.43 |
| | | Polysorbate-60 | 1.51 | 3 | 3.24 | 1.51 |
| | | Beheneth-20 | 0.86 | 1.7 | 1.84 | 0.43 |
| (D) | (d1) | Isostearyl alcohol | 1 | 1 | 1 | 1 |

| | | | | |
|---|---|---|---|---|
| Viscosity (mPa · s) | 7500 | 12200 | 13800 | 4200 |
| Whiteness | S | S | S | S |
| Watery feel | S | S | S | S |
| High-temperature stability | B | A | A | C |

From Table 2, the blending amount of the surfactant (c1) that dissolves when mixed with the component (a2) at 25° C. is insufficient when it is 2.37% by mass (Test example 2-3), and it is preferably 2.5% by mass or greater, and more preferably 3.0% by mass or greater. There is no limitation for the upper limit of the blending amount; however, improvement in the effect is not so remarkable when the blending amount is greater than 6% by mass.

With respect to the viscosity, high-temperature stability was achieved at 7500 mPa·s or greater (Test example 2-1), and high-temperature stability was insufficient at 4200 mPa·s (Test example 2-3).

When the results of Table 1 are taken into consideration, the viscosity is 7500 mPa·s or greater, preferably 10000 mPa·s or greater, and more preferably 15000 mPa·s or greater.

1. An oil-in-water emulsion lip cosmetic comprising the following (A) to (D), and having a viscosity of 7500 to 50000 mPa·s.
(A) An oil component comprising (a1) and (a2)
(a1) 5 to 70% by mass of methylphenylsilicone
(a2) 5 to 70% by mass of an oil component that separates when mixed with (a1) at 25° C.
(B) An aqueous component comprising (b1) and (b2)
(b1) 10 to 80% by mass of an aqueous component
(b2) 0.01 to 10% by mass of a colorant
(C) A surfactant comprising (c1) and (c2)
(c1) 2.5% by mass or greater of a surfactant that dissolves 1% by mass or more relative to (a2) when mixed with (a2) at 25° C.
(c2) 0 to 0.5% by mass of a sucrose fatty acid ester
(D) A higher alcohol comprising (d1)
(d1) 0.5% by mass or greater of a higher alcohol having a branched chain and 6 or more carbon atoms in the total amount of the higher alcohol.

2. The cosmetic of claim 1, wherein the oil component (a2) that separates when mixed with (a1) at 25° C. is preferably selected from a group consisting of a hydrogenated polyisobutene, hydrogenated polydecene, Vaseline, pentaerythritol ester, liquid paraffin, squalane, polyethylene wax, and microcrystalline wax.

3. The cosmetic of claim 1, wherein the alcohol (d1) having a branched chain and 6 or more carbon atoms is isostearyl alcohol.

4. The cosmetic of claim 1, wherein the surfactant (c1) that dissolves when mixed with (a2) at 25° C. is selected from a group consisting of sorbitan monostearate, polyoxyethylene sorbitan fatty acid ester, polyoxyethylene behenyl ether, PEG-40 stearate, PEG-55 stearate, glyceryl stearate, sorbitan sesquioleate, and PEG-5 glyceryl stearate.

5. The cosmetic of claim 2, wherein the alcohol (d1) having a branched chain and 6 or more carbon atoms is isostearyl alcohol.

6. The cosmetic of claim 2, wherein the surfactant (c1) that dissolves when mixed with (a2) at 25° C. is selected from a group consisting of sorbitan monostearate, polyoxyethylene sorbitan fatty acid ester, polyoxyethylene behenyl ether, PEG-40 stearate, PEG-55 stearate, glyceryl stearate, sorbitan sesquioleate, and PEG-5 glyceryl stearate.

7. The cosmetic of claim 3, wherein the surfactant (c1) that dissolves when mixed with (a2) at 25° C. is selected from a group consisting of sorbitan monostearate, polyoxyethylene sorbitan fatty acid ester, polyoxyethylene behenyl ether, PEG-40 stearate, PEG-55 stearate, glyceryl stearate, sorbitan sesquioleate, and PEG-5 glyceryl stearate.
